(19) United States
(12) Patent Application Publication
(10) Pub. No.: US 2007/0162137 A1
(43) Pub. Date: Jul. 12, 2007

(54) Intervertebral disk implant

(76) Inventors: Henning Kloss, Schweiz (DE); Killian Kraus, Werneck (DE); Bjorn Schafer, Ruppichteroth (DE)

Correspondence Address: AMIN, TUROCY & CALVIN, LLP, 1900 EAST 9TH STREET, NATIONAL CITY CENTER, 24TH FLOOR,, CLEVELAND, OH, 44114, US

(21) Appl. No.: 10/585,237

(22) PCT Filed: Dec. 31, 2004
(86) PCT No.: PCT/DE04/02839

§ 371 Date: Jun. 29, 2006

Related U.S. Application Data
(60) Provisional application No. 60/534,344, filed on Jan. 6, 2004.

(30) Foreign Application Priority Data
Dec. 31, 2003 (DE) 103 61 772.8

(51) Int. Cl. A61F 2/44

(52) U.S. Cl. 623/17.15; 623/17.16

(57) ABSTRACT

The invention relates to intervertebral disc implants that imitate the natural freedom of movement and facilitate translational and/or rotational displacements of the intervertebral disc in relation to the base plate, independently of the possible displacements of the base plate in relation to the intervertebral disc. To achieve said displacements, the intervertebral disc is mounted on the base plate by means of the fixing elements located in the interior of the implant, in such a way that translational and/or rotational displacements can take place. In addition, the bearing surface between the top plate and the intervertebral disc is spherical, thus maximising the contact surface.

The present invention relates to an artificial intervertebral disk designed in such a way that the degrees of freedom of movement of a natural intervertebral disk are imitated in the best possible manner.

The spine represents the physical center of movement of the human body. It carries the weight of the body, is able to perform complex movements, and can absorb and compensate the forces acting on it.

The human spine consists of altogether 24 vertebrae, the sacrum and the coccygeal bone. The individual vertebrae are separated by intervertebral disks. The spine is divided into five sections, namely the cervical spine (7 cervical vertebrae, C1-C7), the thoracic spine (12 thoracic vertebrae, Th1-Th12), the lumber spine (5 lumbar vertebrae, L1-L5), the sacrum and the coccygeal bone.

Each vertebra consists of an osseous vertebral body, a vertebral arch spanning the spinal marrow, a transverse process on each side, and a spinous process directed to the rear.

In the medical special disciplines surgery, orthopedics and neurosurgery, the artificial intervertebral disk replacement of traumatically, rheumatically or degeneratively changed spines is one of the operative procedures.

According to prior art, the spine is stiffened in the stressed region. With the help of plate or rod materials, painful regions are bridged which stiffen in the course of time due to a lack of exercise. The stiffening is commonly performed ventrally (situated toward the belly, on the belly side) on the vertebral bodies, or dorsally (belonging to the back, situated toward the back) in the region of the vertebral arches (pedicles).

For the artificial replacement of the intervertebral disk, the endogenic material (annulus fibrosus and nucleus pulposus) is removed by surgery, and a substitute is inserted instead. In most cases, rigid cages are used here, which, depending on the system, are filled with bone cement or with bone chips.

It is disadvantageous in the known systems that a stiffening/fusion of the respective segment of movement is accepted for treatment of the symptoms. A restoration of the spine with respect to form and function is not achieved. The results of such operations are limited movability and the ‘adjacent-disc-syndrom’ (intervertebral disk disease of the intervertebral disk compartment adjacent to a fusion caused as it is overstrained as a result of the fact that it has to bear the resulting forces of movement from the stiffened segment as well).

In recent years, systems have been created which intend to maintain the movability of the vertebral body segments, avoiding a stiff connection of the two vertebrae in the region of the damaged intervertebral disk. Systems of this kind mainly use viscous or deformable material surrounded by a rigid outer covering.

US 2002/0128715 A1, for example, discloses an artificial intervertebral disk consisting of a deformable, elastic inner body which can be deformed within certain predefined limits and is surrounded by a rigid outer skeleton. By means of this artificial intervertebral disk, the natural degrees of freedom of movement are achieved by a predefined limited deformation of the inner body.

What should be improved in all known artificial intervertebral disks is the imitation of the possibilities of movement of a natural vertebral segment. Until now, it has not been possible to provide an artificial intervertebral disk implant with the degrees of freedom of movement shown by a natural vertebral segment. By the insufficient functioning of known implants, the movability of the spine is not restored in an optimal manner. Peak loads occurring during motion which cannot be compensated provoke the sinking of the implants into the vertebral body. In addition to that, in known systems, the problem arises that they are either not stable with respect to loads and cannot cope with the permanent loads acting on the spine, or that the materials do not meet the requirements with respect to biocompatibility. Moreover, the behavior of growing on is still insufficient, and these processes can cause a pressure acting onto the nerve root once again.

It is the object of the present invention to provide an intervertebral disk implant which achieves a maximum of anatomic compatibility and imitates the degrees of freedom of movement of a natural intervertebral disk in the best possible manner even under permanent load, and can thus permanently replace a natural intervertebral disk.

Said object is solved by providing an intervertebral disk implant according to claims 1 and 2, respectively, and use thereof according to claim 14. Further advantageous embodiments, aspects and details of the invention are evident from the dependent claims, the description, the examples and the figures.

The present invention relates to an intervertebral disk implant characterized in that during a rotational movement and/or bending movement the joint center of gravity can be varied in the same manner as in the case of a natural vertebral segment.

The complex movement of a vertebral segment can be shown by the travel of the instantaneous center of rotation (ICR), for example. As the intervertebral disk implants of the invention imitate the natural degrees of freedom of movement in the best possible manner, the invention can be simply expressed in that the intervertebral disk implants according to the invention allow the movements which are possible in the case of a natural vertebral segment.

As, apart from the rotational movement, a natural vertebral segment also allows translational movement, it is now necessary to describe these processes of movement by means of physical quantities. One of these physical quantities is the instantaneous center of movement or travel of the instantaneous center of movement.

According to the invention, the intervertebral disk implants described herein allow travel of the instantaneous center of movement in the same manner as it is possible in the case of natural vertebral segments, which, however, is impossible in the case of the intervertebral disk implants of the prior art.

The degrees of freedom of movement of a vertebral joint are diverse, resulting in complex possibilities of movement and complex patterns of movement. The movement of a vertebral segment can be described as direct rotational movement around and direct translational movement along a spatial axis, the so-called IHA (Instantaneous Helical Axis).

With the possible flexion-extension movements, bilateral side inclination movements as well as rotational movements, paradox patterns of movement of different intensities are created throughout here.

In the case of these structures of movement of the segments in the cervical region, the region of the thorax and the lumbar region, as parameters of the instantaneous position of the helical axis (IHA), angle of rotation, direction and position of the IHA as well as helical pitch have to be considered. Basically, the segmental movability can be shown by a direct helical surface. The outer parameters of the force system, however, can be held constant as functions of time, namely force, torque, direction and position of the line of force effect.

If the position or travel of the IHA during a flexion-extension, bilateral side inclination and/or rotational movement is determined, the curve shown in FIG. 2 for an L3/4 vertebral segment is created, for example.

The axial rotation of the flexional vertebral segment is limited kinematically. The sagittally placed joints produce a mechanical guide forcing the IHA to travel to the rear (see FIG. 2) with increasing rotation. The geometrical moment of inertia relative to the IHA and thus the rotational rigidity of the vertebral segment thus increases, so that a further increase of the torque causes a decreasing angle increase. In the case of flexion, the IHA goes from one joint to another in a ventral bow (see curve course 1 in FIG. 2), whereas in the case of extension the IHA travels on a dorsal bow (see curve course 2 in FIG. 2). Distances of travel of 40 mm to more than 60 mm can be traveled here. After resection of the joints, the IHA is once again situated in the intervertebral disk center (see black area at numeral 3 in FIG. 2).

The initial vertebral segment stiffness (for axial angle of rotation α=0) is set by the flexion/extension position of a sufficiently high axial preload: extension (see curve course 2 in FIG. 2) is accompanied by a flat rotational angle torque [α(T)], and flexion leads to a steep α(T). Shifting of the line of action to posterior stiffens the segment without the necessity of changing the amount of preload as the changed guide of the joints displaces the initial IHA to dorsal and increases the geometrical moment of inertia.

An increasing axial rotation causes an increasing compressive load on the leading vertebral joint. As in the case of a large axial angle of rotation α the IHA travels along the loaded joint, nature has solved the problem of friction kinematically, as the articular surfaces now roll. Sticking friction cannot occur in the case of return of motion, and rolling friction is smaller than sliding friction (M. Mansour, D. Kubein-Meesenburg, St. Spiering, J. Fanghänel, H. Nägerl BIOmaterialien, 2003, 4 (3), 229).

Such kinematic movements cannot be enabled by the conventional intervertebral disk implants of the prior art. However, according to the invention, the intervertebral disk implants described herein allow such translational movements. Accordingly, the intervertebral disk implants according to the invention allow movement of the IHA in the same way as it is the case with a natural intervertebral disk.

The travel which is possible in the case of a natural vertebral segment is achieved in the embodiments of the invention by seating the intervertebral disk on the base plate in a translationally movable manner.

In the intervertebral disk implants of the invention, the helical axis (IHA) can travel in the same way as in the case of a natural vertebral segment. Accordingly, in the case of the intervertebral disk implants of the invention, the helical axis (IHA) can travel along a ventral or dorsal bow.

The IHA considers the translational and rotatory motions with a steady change in the center of motion (ICR: Instantaneous Center of Rotation) and can thereby describe the movement continuously. The recording of the segmental movements between two rigid vertebral bodies with the IHA thus enables the representation of the true axis of rotation. This is a way of visualizing the complex three-dimensional movements.

If the centrode pattern or the pattern of travel of the ICR (ICR: Instantaneous Center of Rotation) in the case of a vertebral segment is examined, the paradox pattern of movement shown in FIG. 5, for example, results for the instantaneous center of motion. The thick dots and the connecting lines positioned there between indicate the travel of the center of rotation here.

As a result of the inventive design of the intervertebral disk implants of the invention, the same patterns of movement as those in the case of a natural vertebral segment are also possible in the case of the artificial vertebral segment of the present invention. This best possible imitation of the natural patterns of movement, i.e., the centrode pattern, is made possible by the seating of the intervertebral disk on the base plate in accordance with the invention.

In the case of a body performing rotational and translational movements in a plane, ICR (Instantaneous Center of Rotation) refers to the instantaneous position of the center of rotation for a certain frozen state.

Observing a planar rotational movement, i.e., the rotational movement of a planar body in a plane, the movement of the individual portions of said planar body can be shown as a rotational movement about an axis of rotation extending perpendicular to said plane. Said axis of rotation intersects the plane in a certain point, the ICR. The spatial positions of certain points on this planar body can now be defined by their velocities, for example. If, for example, the velocity of two points A and B is known and said two points are not located upon each other (see FIG. 4a), the ICR can be determined by laying a straight line perpendicular to the velocity vector of point A [v(A)] through point A, and a second straight line perpendicular to the velocity vector of point B [v(B)] through point B, and determining the point of intersection of the two straight lines. The point of intersection of the two lines is the ICR.

If the velocity vectors v(A) and v(B) extend perpendicular to the vector AB and the lengths of both velocity vectors are known, one obtains the ICR in the point of intersection of the vector AB with the straight line extending through the two extreme values of the two velocity vectors (see FIG. 4b).

Moreover, for a body performing rotational and translational movements in a plane, IAR (Instantaneous Axis of Rotation) refers to an axis around which the body rotates in the case of an instantaneous way of observation where no translation is performed.

The instantaneous center of rotation (ICR) shows a characteristic course in the case of flexion and extension, as shown by FIG. 5, for example. The paradox accompanying rotation in the case of, for example, a rightward inclination of a vertebral segment normally leads to a left-hand rotation, wherein the spinous processes are displaced to the right.

The intervertebral disk implants according to the invention enable such travel movements of the instantaneous center of rotation (ICR) as they are performed by a natural vertebral segment as well, so that the invention consists in the provision of intervertebral disk implants which enable the same travel movement of the ICR as it is performed in the case of a natural vertebral segment.

Said travel movement is enabled by movably seating the intervertebral disk on the base plate, which is described in detail below.

The artificial intervertebral disk according to the invention is preferably constructed in three parts. The middle piece of the intervertebral disk implant is formed by an intervertebral disk, which is preferably seated on the base plate in such a way that both translational movements and rotational movements are possible.

Said translational and/or rotational movements of intervertebral disk relative to the base plate are independent of the possible movements of the cover plate relative to the intervertebral disk. Accordingly, all three parts of the intervertebral disk implant according to the invention can be moved relative to each other, whereby the natural degrees of freedom of movement of the spine can be imitated in the best possible manner.

The seating of the intervertebral disk on the base plate in such a way that translational movements of intervertebral disk and base plate relative to each other are possible can be realized in different ways.

One way of realization comprises the use of fixing means. As fixing means, journals, bulgings, holding devices, pins, flanges and the like as well as other conceivable means for limiting the translational movement of the intervertebral disk on the base plate can be used, which means are preferably mounted on the base plate.

The base plate can have a preferably centrically arranged guide and/or accommodation pin, which extends in the direction of the axis of torsion. Instead of the centric positioning, the guide and/or accommodation pin can also be mounted non-centrally, for example in a dorsally or ventrally displaced manner. Said pin preferably has a diameter of 2 to 15, preferably of 3 to 12 mm, more preferably of 5 to 10 mm and particularly preferably of 6 to 9 mm, and a height of 1 to 5 mm, preferably of 2 to 4 mm, and particularly preferably of 3 to 4 mm. Moreover, such a pin preferably has a cylindrical shape or conical shape, wherein generally ellipsoid shapes can be used, however. An individual pin should be placed substantially centrically on the base plate.

According to the invention, the intervertebral disk has a recess suitable for accommodating the pin or the fixing means, wherein said recess should have a diameter larger than that of the pin. Such a recess preferably has a design ranging from O-shaped to ellipsoid, but can also have a circular design. In the case of the O-shaped or ellipsoid design, the radius in a lateral direction is smaller than the radius in an anteflexion and retroflexion direction.

Preferably, the length of the radius of the recess in an anteflexion and retroflexion direction is one to three times the length of the radius of the pin. The radius of the recess in a lateral direction is, compared to the radius of the pin, of the same size or larger up to two times the radius of the pin.

Due to the larger design of the recess in the intervertebral disk as compared to the pin of the base plate, said pin can move within the limits defined by the recess, or rather, said intervertebral disk can move within said limits in a translational manner on the base plate.

Expressing these relations in absolute figures, the intervertebral disk can preferably move 0 to 10 mm, preferably 1-6 mm, more preferably 2-5 mm, and particularly preferably 3-4 mm in a lateral direction, and 2 to 15 mm, preferably 3-10 mm, more preferably 4-7 mm, and particularly preferably 5-6 mm in an anteflexion direction as well as in a retroflexion direction on the base plate. Said figures refer to the total distance from one extreme position to another. The half-lengths are traveled from a centered position to an extreme position.

A fixing means in the form of a pin substantially centrically mounted on the base plate does not limit rotational movement of the intervertebral disk on the base plate, however. The rotational movement around the axis of torsion is determined by the natural conditions and/or by further fixing means, however. Such fixing means are preferably mounted on the base plate. If rotation is not technically limited on the implant, free rotation is limited by the physiologically existing structures, of course. The intervertebral disk implant according to the invention allows rotational movements of up to 3 degrees, preferably of 1-2 degrees, and particularly preferably of about 1.5 degrees in both directions.

The fixing means can not only consist of one pin, journal, flange or the like, but can also comprise two or more of these fixing means. Especially fixing means are preferred which are completely covered by the intervertebral disk. Lateral limitations in the form of, for example, edges, holding devices, beads, rails or the like disposed on the base plate or at the edge of the base plate are less preferred as these represent a point of adsorption for tissue and can be overgrown by tissue and/or cartilage whereby the motility of the intervertebral disk on the base plate is limited again. Thus, especially fixing means are preferred which are completely covered, i.e. are not accessible by tissue, cartilage and muscles. The fixing means are completely covered if they, for example, are located in the interior of the implant, for example, are covered by the intervertebral disk.

A further preferred embodiment comprises two pins which are mounted on the base plate in a preferably dorsally or ventrally offset manner. The intervertebral disk correspondingly has two recesses having a larger diameter compared to the diameter of a pin. Thereby, the intervertebral disk can freely move around the pins in a translational manner within the recesses, wherein translational movement as well as rotational movement around the mechanical or anatomical axis is possible within the bounds of the recesses. In the case of this embodiment, a theoretically possible free rotation by 360 degrees can no longer be performed.

Instead of two pins, three or more can also be used, which are, as a rule, equidistantly mounted on the base plate. Furthermore, instead of pins, lateral holding devices can also be provided. In this case, the surface area of the base plate limited by the holding devices which are laterally fixed on the base plate is made larger than the surface area of the intervertebral disk lying thereon, so that the intervertebral disk can perform translational and/or rotational movements on the base plate or relative to the base plate within the bounds of the lateral holding devices. Such holding devices can be a continuous or discontinuous bead at the edge or a raised edge, for example.

It is not necessary for the intervertebral disk to have a round or cylindrical shape as shown in FIGS. 7 and 8, but instead it can have desired common designs ranging from oval to cornered, angular to banana-shaped, flat to hunch-shaped, asymmetrical to square or rectangular. Furthermore, the intervertebral disk can be tapered, i.e., vary in thickness, and can be tapered in a dorsal direction in particular. Possible basic shapes of an intervertebral disk are disclosed in European Patent EP 0 505 634 B1 as FIG. 2 and FIG. 3(a)-(e), for example. The possible basic shapes of the intervertebral disk furthermore do not have to have a uniform thickness, so that different locations of the intervertebral disk can also have different thicknesses, which can be 3 mm, 6 mm, 9 mm or 12 mm, for example. Moreover the intervertebral disk is preferably non deformable.

A preferred embodiment comprises intervertebral disk implants in which the cover plate is seated on the intervertebral disk in such a manner that the articulating surface of the intervertebral disk and the articulating surface of the cover plate are each situated on a respective ellipsoid partial surface.

“Articulating surface” refers to the surface of the intervertebral disk or the surface of the cover plate which can contact the corresponding other surface with the possible movements.

Said articulating surfaces of the intervertebral disk and the cover plate which come into contact with each other are located on parts of the surfaces of ellipsoid bodies, preferably spheres.

It is intended that the contact surface refers to the area where, in a certain frozen position of the intervertebral disk and the cover plate, the two parts come into contact with each other.

In contrast to that, the articulating surface of the intervertebral disk is the entire surface of the intervertebral disk which can come into contact with the surface of the cover plate in any possible positions of the intervertebral disk relative to the cover plate.

Accordingly, the articulating surface of the cover plate is the entire surface of the cover plate which can come into contact with the surface of the intervertebral disk in any possible positions of the cover plate relative to the intervertebral disk. According to the invention, the articulating surface of the cover plate is located on a partial surface of an ellipsoid, preferably on a compressed (a=b>c) ellipsoid of revolution or an elongated (a=b<c) ellipsoid, and particularly preferably on a spherical surface section (a=b=c). Therein, a refers to the radius in the direction of the x-axis (anteflexion-retroflexion axis), b to the radius in the direction of the y-axis (axis of torsion), and c to the radius in the direction of the z-axis (lateral axis). The same applies to the articulating surface of the intervertebral disk in a corresponding manner.

It is further important that, according to the invention, the radii (a, b and c; or a and c; or a) of the ellipsoid surface or the spherical surface on which the articulating surfaces of the cover plate are located have the same sizes as the radii (a′, b′ and c′; or a′ and c′; or a′) of the ellipsoid surface or the spherical surface on which the articulating surfaces of the intervertebral disk are located.

Particularly preferably, the articulating surface of the cover plate is located on a spherical surface section and the articulating surface of the intervertebral disk is also located on a spherical surface section, wherein further both spherical surface sections particularly preferably have the same radius.

The radii of said spherical surface sections on which the articulating surfaces of intervertebral disk and cover plate are located have dimensions of R=15-45 mm. Depending on the size of the intervertebral disk implant, the radii also increase correspondingly. Intervertebral disk implants for the lumbar region have radii of 25-45 mm, for the thoracic region of 20-40 mm, and for the cervical region of 1.5-35 mm.

The contact surface is an area of 400 mm² at least, preferably of at least 450 mm², more preferably of at least 500 mm², and particularly preferably of at least 550 mm². It also has to be taken into consideration here that the contact surface depends on the size of the implant and larger intervertebral disk implants thus have larger contact surfaces. Contact surfaces of this size distribute the mechanical load on the intervertebral disk and result in a longer durability of the implant.

By means of the design according to the invention, the contact surface between the cover plate and the intervertebral disk is maximized even in the case of complex movements as not a punctual or line-shaped contact surface, but instead a spherical contact surface is created.

Basically, two embodiments are conceivable therefor. According to the first possibility, the articulating surface of the cover plate can be designed convexly or plano-convexly, and the surface of the intervertebral disk articulating with the cover plate concavely or plano-concavely (see FIG. 10); or else, the articulating surface of the cover plate is designed concavely or plano-concavely, and the surface of the intervertebral disk articulating with the cover plate convexly or piano-convexly (see FIG. 11). The embodiment mentioned first is preferred here.

It is further particularly preferred that in the case of a concave design of articulating surface of cover plate or of intervertebral disk the contact surface corresponds to the articulating surface. In this embodiment, the radii of the spherical surface sections on which the articulating surfaces of intervertebral disk and cover plate are located are substantially identical.

A preferred embodiment of the intervertebral disk implant thus comprises a base plate, an intervertebral disk and a cover plate, wherein said intervertebral disk is seated on the base plate in such a manner that translational and/or rotational movements are possible, and said cover plate is seated on the intervertebral disk in such a manner that the articulating surface of the intervertebral disk as well as the articulating surface of the cover plate are each located on a respective ellipsoid partial surface, preferably a spherical surface.

In the embodiments according to the invention, furthermore, the cover plate can be inclined by up to 20 degrees relative to the base plate starting from a parallel position with respect to each other.

Further, tapering cover and base plates such as those shown in FIG. 10 are preferred. On their ventral sides, cover and base plates are thicker than on their dorsal sides in order to imitate the natural shape of a vertebral segment in a better way. Cover plate or base plate or cover and base plate have preferably on their ventral side the double thickness as on the dorsal side. Alternatively, the cover plate or base plate or cover and base plate have an inclination of 3%, preferred 6%, and especially preferred 8% from the dorsal to the ventral end. It is especially preferred if the sides of cover plate and base plate facing each other have no chamfer and only the sides of cover plate and base plate not facing each other are chamfered. In another preferred embodiment whether only base plate or only cover plate are tapering. The chamfer of the cover plate and/or base plate ca be up to 10 degrees, preferred 2 to 8 degrees (vetrally broad and dorsally tapering off). Further, it is preferred if according to the curvature of the spine the declination of the cover plate and/or the base plate is adapted to the physiological conditions wherein preferably the cover plate has in the cranial (to the head) direction a different degree of the declination than the base plate has in the caudal (to the foot) direction. Viewing from the side, the spine describes a double S (kyphosis/lordosis). Especially in the area of the lumbar spine (lordosis) the vertebrae are in an angle opened to the ventral direction to each other. To ideally serve the corresponding intervertebral section the implant should be adaptable with its cover and base plates to these vertebrae being in an angle to each other. The background therefor is to provide for an ideal force impact on the implant, to minimize the risk of luxation of the intervertebral disk and to adjust the intervertebral structures of the spine according to the physiology. These aspects would add to the durability of the implant. If the intervertebral section is filled out according to the anatomic structures and if the intervertebral disk is loaded ideally the abrasion of the polyethylene of the intervertebral disk is reduced. As an intervertebral disk of polyethylene or also other polymeric materials are subject to abrasion in the most intense way an especially preferred embodiment of the present invention uses an intervertebral disk of metal, optionally covered with a ceramic coating.

Further, it is preferred if the base plate as well as the cover plate have a convex curvature of the surface facing to the bone. Especially preferred is the convex curvature of the cover plates to the bone as also the vertebrae have a curvature (concave) and so a sintering of the implant into the bone can be prevented. An advantage would be that the structure of the bony trabeculum would be loaded according to the physiology, more surface for ongrowth of the bone would be available and that the luxation risk of the implant would be reduced. Thereby, the convexity is preferably in a range of 1-5 mm, i.e. the elevation is up to 5 mm at the highest point.

In addition to the possibility of movement of cover plate and intervertebral disk relative to each other, the intervertebral disk and the base plate can be moved relative to each other. The intervertebral disk implant according to the invention is designed in such a way that the intervertebral disk is seated on the base plate in such a manner that the intervertebral disk can be rotated in the horizontal plane by a few degrees around the axial axis of torsion.

The movement of the base plate and the cover plate relative to each other can be compared to the movement of two identical parallel plates, between which an ellipsoid, in the optimum case a sphere, is located, wherein the respective plate contacts the ellipsoid or the sphere in the center of the plate. The movement of the plates relative to each other can be compared with the movement of the base plate and the cover plate of the intervertebral disk implant according to the invention with respect to each other, wherein, due to the design of the base plate and the cover plate, a lateral bending movement as well as a retroflexion movement can only be performed to a smaller extent than an anteflexion movement.

The base plate and the cover plate can be rotated relative to each other by a maximum of 10 degrees, preferably up to 8 degrees, more preferably up to 6 degrees, and particularly preferably up to 4 degrees.

A bending movement in a lateral direction can be performed by up to 8 degrees, preferably up to 12 degrees, and particularly preferably up to 15 to both sides starting from a centered position.

A retroflexion bending movement can be performed by up to 10 degrees, preferably up to 15 degrees, and particularly preferably up to 20 degrees starting from a centered position.

An anteflexion bending movement can be performed by up to 20 degrees, preferably up to 25 degrees, and particularly preferably up to 30 degrees starting from a centered position. Further, the intervertebral disk is preferably designed such that it has a size such that in all of the possible movements the cover plate does not contact the base plate. Further, the edges of cover and base plate have a declination (see FIG. 10) of the corners facing each other away from each other. This declination of the edges of base and cover plate as well as the design of the intervertebral disk are substantial for a long operability of the implants according to the invention as the contacting resp. grinding (impingement) of cover and base plate can result in an abrasion and in the release of particles up to bigger implant pieces which drastically reduce the durability of the implant. Moreover, the case of luxation of the intervertebral disk can occur when base and cover plate contact each other and the contact area between cover plate and intervertebral disk is reduced due to an elevated cover plate. Thus, due to the reasons mentioned before, an impingement of cover and base plate is to be prevented necessarily.

Further, the intervertebral disk can preferably be made of a rigid plastic, preferably polyethylene, and in particular ultra high molecular weight polyethylene (UHMWPE).

The designation “ultra high molecular weight polyethylene” is not definitely clear. HDPE (high density PE) currently refers to a PE having a molar weight of less than 200,000 g/mol. According to DIN ISO 11542, PE having a melt mass flow rate of less than 0.1 g/10 min is defined as UHMWPE (which would correspond to a molar weight of more than 106 g/mol), according to ASTM D 4020, the limit is 3.1*106 g/mol. The indicated mean molar weight of current UHMWPE is between 3.5*106 and 107 g/mol, depending on the manufacturer and the measuring method used. Ultra high molecular weight polyethylene (UHMWPE) is a polyethylene according to ISO 5834-2 Standard, Chirulen® and TIVAR® Premium are high-purity implant materials of PEUHMW for use in endoprosthetics. As preferred articulation partners, they are used in artificial hip, knee, elbow and shoulder joints.

In further preferred embodiments, titanium or a titanium alloy is also used to make the intervertebral disk. In these further preferred embodiments having a base plate of titanium or a titanium alloy, a cover plate of titanium or a titanium alloy as well as an intervertebral disk of titanium or a titanium alloy, so-called hard-hard pairs are created between the cover plate and the intervertebral disk and also between the base plate and the intervertebral disk. In these systems it is further particularly preferred if the titanium or the titanium alloy is provided with a ceramic coating. Basically, embodiments are preferred which do not use a polymer but a metal or a metal alloy for the intervertebral disk which is preferably provided with a ceramic coating.

The titanium materials approved for medicinal engineering should meet with DIN ISO 5832-3 in particular. In principle, the approval of titanium and titanium alloys as a medicinal material is regulated by the DIN ISO 5832-1 to 5832-12 Standards.

Apart from pure titanium, also titanium alloys such as Ti-6Al-4V, Ti—Nb—Ta—Zr, Ti—Al6-Nb7 (according to ISO 5832-11) or Ti-29Nb-13Ta-4.6Zr can thus be used according to the invention. Preferred are titanium alloys in which the titanium portion is at least 50% by weight, more preferably 65% by weight, even more preferably 80% by weight, and particularly preferably 90% by weight. Further, the use of pure or medical titanium for making the entire intervertebral disk implant is preferred.

Base and cover plates can be cemented, or implanted into the bone or fixed to the vertebral bone without cement, wherein the anchoring without cement is preferred.

Further, titanium is used as material for the basic body of the base plate and/or cover plate. Titanium as basic material of the base and cover plates according to the invention is biologically inert, thus fixedly grows together with the bone, can be anchored without any cement, and is anallergic.

By selecting biocompatible, inert materials, the acceptance of the physiological tissue onto the implant is improved essentially. Due to the use of materials which are especially suitable for withstanding tribological stresses, wear of the artificial material is minimized and, accordingly, the durability (service life) of the implant is prolonged essentially.

Bone cells can directly anchor onto biocompatible materials if a structured surface having an open roughness in the range of 50 to 400 μm is provided.

To enable the base plate and the cover plate to fixedly grow together with the bone especially in the case of the fixing without cement, the surfaces of the base plate and the cover plate facing the bone have a roughness of at least Rz 50 μm, preferably at least Rz 60 μm. Of course, also other degrees of roughness up to spongiosa metal can be used.

The roughness is either indicated as Rz or Ra (DIN 4762, 4768, 4775, ISO 4288). Rz refers to the mean depth of roughness. The mean depth of roughness Rz is the arithmetical mean of the largest individual depths of roughness of several individual measuring distances which are adjacent to each other. In contrast to that, Ra refers to the arithmetical average height. Ra is the generally acknowledged and internationally used roughness parameter. It is the arithmetical mean value of the absolute values of the profile variations within the reference distance. The measured numerical value Ra is always smaller than the Rz value determined on the same roughness profile.

The base plate and/or the cover plate of the intervertebral disk implant according to the invention are preferably coated with a metallic or ceramic coating, which can have a variable number of individual layers or coats, or a varying layer or coat thickness. Ceramic coatings comprise nitrides, carbides and phosphides of preferably semimetals and metals or metal alloys. Examples of ceramic coatings are boron nitrides, titanium-niobium-nitride, titanium-calcium-phosphide (Ti—Ca—P), Cr—Al—N, Ti—Al—N, Cr—N, TiAlN—CrN, Ti—Al—C, Cr—C, TiAlC—CrC, Zr—Hf—N, Ti—Hf—C—N, Si—C—N—Ti, Si—C—N as well as DLC (Diamond Like Carbon). A ceramic coat or layer of titanium-niobium-nitride (Ti—Nb—N) is further preferably applied as coating.

It is particularly preferable if the articulating surfaces of the base plate and of the cover plate are coated with titanium-niobium-nitride (Ti—Nb—N).

This ceramic coating of the particularly articulating implant surfaces has a hardness which is many times higher than that of commonly used materials. As a result of this hardness, the surface is highly polishable and protected from titanium abrasion.

According to the invention, the geometry of the articulating compartments is selected in such a way that the surfaces which are subject to wear can be maximized. This means that, according to the invention, the geometry of the joint partners is selected in such a way that the tribologically stressed areas are maximized via a plane contact surface between the cover plate and the intervertebral disk, and an ellipsoid surface section, preferably a spherical section, between the intervertebral disk and the cover plate, which in the end reduces wear. This results in a reduction in the forces acting per unit of area, which, in turn, has a positive effect on the service life of the implant as a result of a reduction in the abrasion. By the selected and individually adapted geometry of the intervertebral disk implant, in particular the geometry of the intervertebral disk, and the correct positioning of the implant during surgery, a correspondence with the physiological movability of the vertebral body segments with respect to each other is achieved in the best possible manner. By means of this nearly perfect imitation of a natural intervertebral disk or its movability, the forces acting on the bone-implant-boundary are reduced considerably, which has a positive effect on the longevity (reduced wear and minimization of loosening processes) of the implant.

The prostheses of the prior art can mostly only be inserted on maximally two levels in the back. The intervertebral disk implants according to the invention can also be inserted on more than two levels in the backbone. In this case, the individual intervertebral disk implants are adapted with respect to size and geometry to their respective positions, so that also spine ailments, spine damage as well as diseases of the spine can be treated by means of such multiple implants.

Said spine ailments, spine damage as well as diseases of the spine, which can be treated by an intervertebral disk implant according to the invention or by a set of intervertebral disk implants according to the invention, include, for example, scoliosis, i.e., lateral curvature of the spine, also called curvature of the backbone, herniation of intervertebral disk, which refers to the prolapse of the core of the intervertebral disk against the adjacent vertebral bodies or the nerve roots, as well as kyphosis, which means curvature of the spine to the rear.

Further, by the intervertebral disk implants according to the invention, the following spine ailments, spine damage as well as diseases of the spine can be treated: Disk rupture (i.e., intervertebral disk disease), Black Disc (degenerative intervertebral disk occurring black in the X-ray picture), spontaneous deformation, i.e., the deformation of vertebral bodies by diseases, bone changes or swellings, lumbago, or more commonly referred to as lumbar rheumatism or lumbar pain, which refers to a severe, in most cases suddenly occurring pain in the back and lumbar regions. Lumbago most commonly results from intervertebral disk changes. Spondylosis deformans, i.e., disease of the vertebral bodies and intervertebral disks with severe pain on motion, age-related hunchback (Witwenbuckel), i.e., a curvature of the spinal column of elderly women caused by bone atrophy due to the changed hormonal situation after the climacterium (menopause), spondylomyelitis, i.e., inflammation of vertebrae and the spinal cord, osteochondrosis, which refers to changes in and degeneration of intervertebral disks, as well as osteofibrosis, which designates the diseases of the skeleton of juveniles, spina bifida, also known as cleft vertebra, in particular the innate cleft formation of the spine, lordosis, which, for an expert, means a forward curvature of the spine caused by a hollow back, spondylotosis, i.e., slippage of a vertebral body over an entire vertebra width, in most cases of the 5^{th} lumbar vertebra onto the sacrum, clay shoveller's fracture designating an avulsion fracture, in most cases of the 7^{th} cervical vertebra or the spinous process of the 1^{st} thoracic vertebra, caused by severe overstrain, myelomeningocele, which is understood by an expert as an innate malformation of vertebral archs, brachialgia, which refers to pains in the arms and shoulders due to changes in the region of the cervical vertebrae, Baastrup's syndrome, which means the forward bending of the spine with a widening of the spinous processes and crushing of the tissue in between, which is in most cases accompanied by severe back pain and pain in the spinous processes on pressure, vertebral ankylosis, which refers to the bony stiffening of the spine with severe pain in the truncus, arms and legs and paralysis of the limb muscles, Scheuermann's disease, which among experts refers to inflammations of bone and cartilage of the individual vertebral bodies, preferably of the thoracic spine of juveniles, cervical syndrome, i.e., diseases of the soft parts in the region of the cervical spine, lumbar kyphosis, i.e., curvature of the spine in the region of the lumbar vertebrae, torticollis, i.e., wry-neck, often based on rheumatism, as well as the Bechterew's disease, which refers to the chronic inflammatory spine disease, which causes changes in and stiffening of the entire spine apparatus.

### DESCRIPTION OF THE FIGURES

FIG. 1 shows two vertebrae with vertebral body, vertebral canal, vertebral arch, transverse process and spinous process as well as the associated intervertebral disks;

FIG. 2 shows a horizontal section through an L3/4 intervertebral disk as well as the IHA travel in the case of a rotational movement. In the case of flexion, the IHA runs in a ventral bow from one joint to another (1), in the case of extension, however, in a dorsal bow (2). The distances of travel can be 40 mm up to >60 mm. After resection, the IHA is located in the center of the intervertebral disk (3) [FIG. 2 and text have been taken from the publication by M. Mansour, D. Kubein-Meesenburg, St. Spiering, J. Fanghänel, H. Nägerl BIOmaterialien, 2003, 4 (3), 229];

FIG. 3 shows the ventral and dorsal sections of a vertebral joint on the levels of L1 to L4 and L5, respectively. It is evident that, on the level of L5, the joint rather extends in a frontal plane. The axial rotation, with about 1.5°, is also higher there than in the other lumbar vertebral segments L1 to L4 with about 1 degree [FIG. 3 and text were taken from the publication by M. Krismer, C. Haid, M. Ogon, H. Behensky, C. Wimmer, Orthopädie 1997, 26, 516-520];

FIG. 4 shows a possibility of determining the ICR (Instantaneous Center of Rotation) by means of vectors for the velocity of two points lying in a plane;

FIG. 5 shows the instantaneous center of rotation (ICR: Instantaneous Center of Rotation) in the case of flexion or extension according to Gertzbein. The thick dots as well as the thick connecting line indicate travel of the center of rotation depending on the movement. [FIG. 5 and text were taken from the publication by M. Krismer, C. Haid, M. Ogon, H. Behensky, C. Wimmer, Orthopädie 1997, 26, 516-520];

FIG. 6 shows the distal base plate of the implant. An anchorage suitable for central accommodation of the intervertebral disk admitting both rotational and translational movements is shown;

FIG. 7 shows the intervertebral disk seen from the side facing the base plate. A round recess suitable for accommodating a fixing means such as a pin mounted on the base plate is shown;

FIG. 8 shows the intervertebral disk seen from the side facing the cover plate. The concavely designed articulating surface of the intervertebral disk is shown. The radii R imply that the surface of the intervertebral disk articulating with the cover plate is located on a spherical surface section;

FIG. 9 shows the cover plate with the surface facing the intervertebral disk with its articulating surface designed in a plano-convex manner. The convex, centrally arranged bulging has the same radius as the articulating surface of the intervertebral disk according to FIG. 8, so that the articulating surface of the cover plate is located on a spherical surface section having the radius R;

FIG. 10 shows an embodiment according to the invention of an intervertebral disk implant;

FIG. 11 shows a further embodiment of an intervertebral disk implant according to the invention.

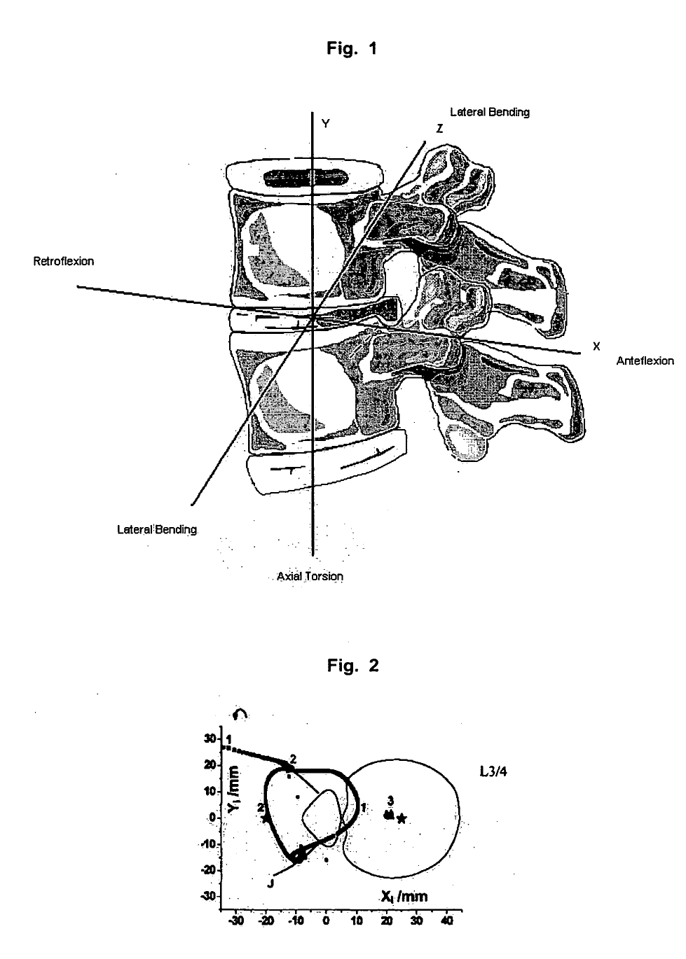

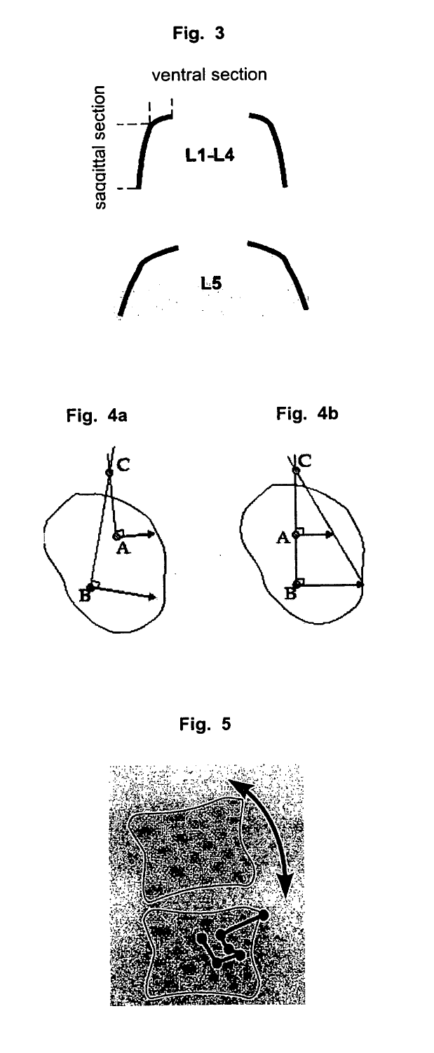

### EMBODIMENTS

Preferred embodiments of the intervertebral disk implant of the invention will now be discussed by means of the examples, wherein it has to be taken into account that the examples discussed show preferred embodiments of the invention, but do not limit the scope of protection to these embodiments.

### EXAMPLE 1

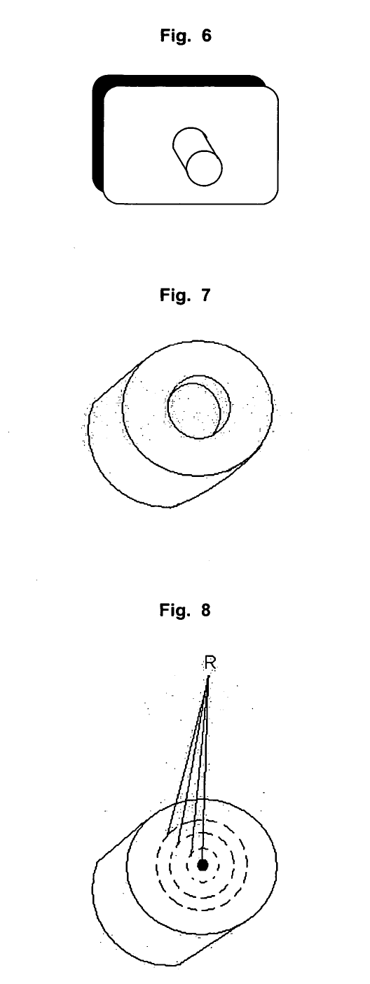

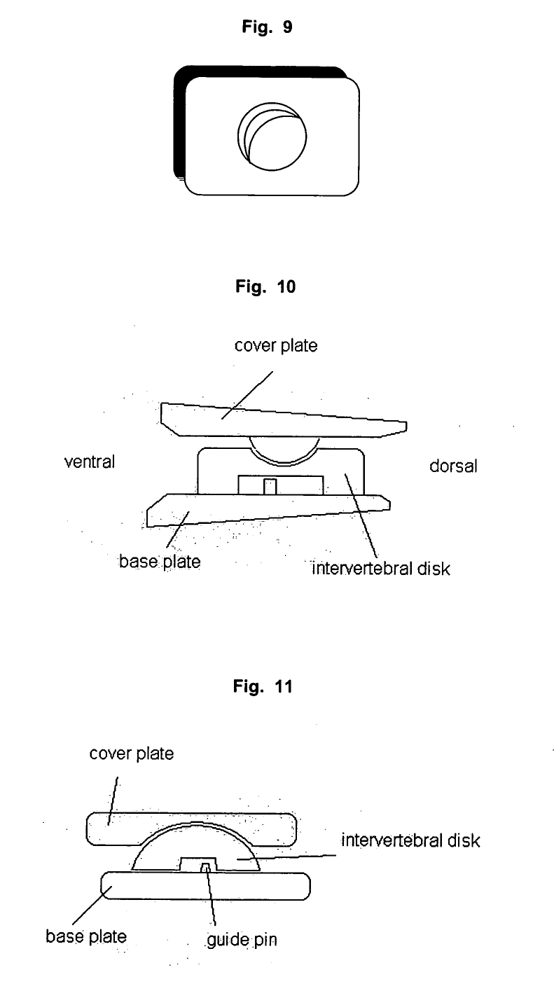

An embodiment of an intervertebral disk implant according to the invention consists of a cover plate as shown in FIG. 9, an intervertebral disk as shown in FIGS. 7 and 8, and a base plate as disclosed in FIG. 6.

The intervertebral disk implant has a size suitable for replacing a L3/4 vertebral segment. Smaller embodiments of the intervertebral disk implant described in example 1 can be produced by a person skilled in the art without difficulty. In the smaller embodiments, the contact surfaces, in particular between the intervertebral disk and the cover plate but also between the intervertebral disk and the base plate, can be correspondingly smaller corresponding to the size of the smaller embodiments. The same applies to the above-mentioned values for the translational movements in a lateral as well as a retroflexion anteflexion direction.

The cover plate consists of titanium used in medical engineering. The surface of the cover plate which is facing the bone is rough, thus enabling the bone cells to grow in or grow on. The roughness Rz is about 60±5 μm. The articulating surface of the cover plate is designed in a piano-convex manner as shown in FIG. 9 and coated with a ceramic coat of Ti—Nb—N. The thickness of the coat is 3-5 μm.

The articulating surface of the cover plate is located on a spherical surface section having a radius of R=25 mm.

The base plate also consists of titanium and has a shape as shown in FIG. 6. The surface of the base plate facing the bone is designed in a rough manner with a roughness Rz of about 60±5 μm. The supporting surface for the intervertebral disk is coated with a ceramic coating of Ti—Nb—N. The thickness of the coat is 3-5 μm.

As evident from FIG. 6, the surface of the tibia component facing the intervertebral disk is planar apart from the centrally positioned pin. Said pin has a cylindrical shape having a height of 5 mm and a diameter of 7 mm. The pin is also coated with a ceramic coating of Ti—Nb—N. The base plate is shown in a rectangular manner, but can, of course, also have other outlines and vary in thickness, as shown by FIG. 10.

The intervertebral disk has a design as shown in FIGS. 7 and 8. FIG. 7 shows the bottom surface of the intervertebral disk with a cylindrical recess for accommodating the guide and/or accommodation pin of the base plate. FIG. 8 shows the top side of the intervertebral disk with its concavely designed articulating surface. The articulating surface is located on a spherical surface section with the radius R. The concentric circles drawn in a dashed manner on the articulating concave surface of the intervertebral disk make clear that this surface is part of a spherical surface. The intervertebral disk consists of UHMWPE. The side of the intervertebral disk facing the cover plate is designed in a concave manner and has a radius of 25 mm. The concave depression of the intervertebral disk with R=25 mm accommodates the convex bulging of the cover plate with also R=25 mm in such a way that a contact surface is created which is located on a spherical surface section. The contact surface created altogether amounts to about 450 mm². Thereby, the load is distributed over an area and not over a point or line on the intervertebral disk.

The entire articulating surface of the concave depression of the intervertebral disk corresponds to the contact surface.

In addition, as shown in FIG. 8, the intervertebral disk has a recess on its side facing the base plate. Said recess is provided for accommodating the pin of the base plate. Said pin is shown in FIG. 6.

Due to the larger diameter of the recess in the intervertebral disk compared to the diameter of the pin of the base plate, the intervertebral disk is able to make both rotational and translational movements on the base plate. The rotational movement is physiologically limited to about 1.5 degrees.

The pin on the base plate has a diameter of 7 mm. The recess in the intervertebral disk has a diameter in a lateral direction of 11 mm and in a retroflexion anteflexion direction of 13 mm. Thus, in a lateral direction, the recess has a diameter which is 1.57 times the diameter of the pin, and in a retroflexion anteflexion direction, the recess has a diameter which is 1.86 times the diameter of the pin.

Proceeding from a central position, the intervertebral disk can move on the base plate 2 mm in a lateral direction, or rather altogether 4 mm from one lateral extreme position to the other. Proceeding from a central position, the intervertebral disk can move on the base plate 3 mm in a retroflexion direction and 3 mm in an anteflexion direction, or rather altogether 6 mm from the dorsal extreme position to the ventral extreme position.

When a bending movement takes place, base plate and cover plate can be inclined by up to 20 degrees relative to each other. Complex movements cause such a travel of the IHA as performed in the case of a natural L3/4 vertebral segment. The same applies to the instantaneous center of rotation (ICR).

Accordingly, the embodiment of the invention allows degrees of freedom with respect to movement just like those existing in the case of a natural vertebral segment, wherein, even in the case of complex movements, load peaks on the intervertebral disk are avoided by the spherical surfaces of intervertebral disk and cover plate lying on top of each other.

### EXAMPLE 2

A further embodiment of an intervertebral disk implant according to the invention for a L2/3 vertebral segment consists of a cover plate, an intervertebral disk and a base plate as disclosed in FIG. 10.

The cover plate consists of titanium alloy Ti—Al6-Nb7 according to ISO 5832-11. The surface of the cover plate which is facing the bone is rough with a roughness Rz of about 55±5 μm. The articulating surface of the cover plate is designed in a plano-convex manner and covered with a ceramic coating of about 6 μm thick. Ti—Ca—P or Si—C—N—Ti or DLC were used as ceramic coating.

The articulating surface of the cover plate is located on a spherical surface section with a radius of R=24 mm.

The base plate also consists of Ti—Al6-Nb7. The surface of the base plate which is facing the bone is rough with a roughness Rz of about 55±5 μm. The supporting surface for the intervertebral disk is covered with a ceramic coating of Ti—Ca—P or Si—C—N—Ti or DLC. The thickness of the coating is about 6 μm. Further, the ground plate has a guide pin with a diameter of 5.5 mm and a height of 4 mm. Also the pin is coated with Ti—Ca—P or Si—C—N—Ti or DLC.

The cover plate has a convex curvature with a maximum elevation of 3.5 mm on its side facing to the bone. Additionally, the cover plate has an inclination degree of 8 degrees and is at its ventral side almost twice as thick as at the dorsal side. Also the base plate has a chamfer of 6 degrees with a form tapering in the dorsal direction.

The intervertebral disk also consists of Ti—Al6-Nb7 according to ISO 5832-11 with a ceramic coating of Ti—Ca—P or Si—C—N—Ti or DLC. The thickness of the coating is about 6 μm.

The intervertebral disk has an oval recess at its bottom side which has laterally a diameter of 7 mm and ventral-dorsally a diameter of 10 mm. The contact surface to the cover plate is about 440 mm².

### EXAMPLE 3

A further embodiment of an intervertebral disk implant according to the invention for a Th5/6 vertebral segment consists of a cover plate, an intervertebral disk and a base plate as disclosed in FIG. 10.

The cover plate consists of the titanium alloy Ti—Al6-Nb7 according to ISO 5832-11. The surface of the cover plate which is facing the bone is rough with a roughness Rz of about 65±5 μm. The articulating surface of the cover plate is designed in a plano-convex manner and coated with a ceramic coating of Ti—Al—N having a thickness of 4 μm.

The articulating surface of the cover plate is located on a spherical surface section having a radius of R=22 mm.

The base plate also consists of Ti—Al6-Nb7. The surface of the base plate facing the bone is designed in a rough manner with a roughness Rz of about 65±5 μm. The supporting surface for the intervertebral disk is coated with a ceramic coating of Ti—Al—N. The thickness of the coat is 4 μm. In addition to that, said base plate has a guide pin having a diameter of 6 mm and a height of 4 mm. The pin is also coated with Ti—Al—N.

The intervertebral disk consists of UHMWPE or of titanium or of Ti—Al6-Nb7 according to ISO 5832-11. If titanium is used as material, the intervertebral disk is entirely, or at least on its articulating surfaces on the lower and upper sides, coated with a ceramic coating of Ti—Nb—N. The thickness of the coat is 3-5 μm. If Ti—Al6-Nb7 is used, a ceramic coating of Ti—Al—N is applied at least onto the articulating surfaces.

The intervertebral disk has an oval recess on the bottom side thereof, said oval recess laterally having a diameter of 7 mm, and ventral-dorsally having a diameter of 12 mm. Accordingly, the intervertebral disk can laterally move 0.5 mm each way, or absolutely travel a distance of 1.0 mm, whereas in a ventral direction a translational movement of 3 mm is possible, and in a dorsal direction also a translational movement of 3 mm is possible, or rather, a distance of 6 mm can be traveled from the dorsal extreme point to the ventral extreme point.

The surface articulating with the cover plate is designed in a concave manner, having a radius of R=22 mm. A contact surface of at least 420 mm² results.

Cover plate and bottom plate slightly taper in a dorsal direction, can be rotated by up to 2 degrees relative to each other and tilted by up to 15 degrees relative to each other.

In the case of these complex rotational movements and bending movements of the intervertebral disk implant, the IHA performs the same travel movements as in the case of a natural vertebral segment. Accordingly, the embodiment of the invention allows degrees of freedom with respect to movement just like those existing in the case of a natural vertebral segment, wherein, even in the case of complex movements, load peaks on the intervertebral disk are avoided by the spherical surfaces of intervertebral disk and cover plate lying on top of each other.

### EXAMPLE 4

A further embodiment of an intervertebral disk implant according to the invention for a C2/3 vertebral segment consists of a cover plate, an intervertebral disk and a base plate as shown in FIG. 11.

The cover plate and the bottom plate consist of titanium alloy Ti-29Nb-13Ta4.6Zr. The surface of the cover plate which is facing the bone is rough with a roughness Rz of about 55±5 μm. The articulating surfaces of cover plate and bottom plate are provided with a coating of Ti—Hf—C—N or Zr—Hf—N having a thickness of about 3 μm.

The cover plate is designed in a plano-concave manner, having a bulge having a radius of 18 mm.

The intervertebral disk consists of UHMWPE or of titanium or of Ti—Al6-Nb7 according to ISO 5832-11 or of Ti-29Nb-13Ta4.6Zr. If titanium or a titanium alloy is used as material, the intervertebral disk is entirely, or at least on its articulating surfaces on the lower and upper sides, coated with a ceramic coating.

The intervertebral disk is designed in a piano-convex manner, having an articulating surface which is located on a spherical surface and which has the same radius as the spherical surface on which the articulating surface of the cover plate is located. The contact surface has a size of at least 400 mm².

The round recess in the intervertebral disk suitable for accommodating the guide pin has a diameter of 6 mm.

The cylindrical guide pin mounted on the base plate has a height of 3 mm and a diameter of 4 mm. Due to these dimensions, the guide pin or the intervertebral disk can horizontally translationally move on the base plate 1 mm to any directions proceeding from a centered position. Rotational movement is possible up to one degree.

The artificial cervical vertebra implant is thus able to carry out such motions as they are performed by the natural C2/3 vertebral segment, and the instantaneous center of rotation (ICR) and the IHA make the same travel movements as in the case of the natural vertebral segment.

### EXAMPLE 5

Materials and designs of the base plate and the cover plate are similar to those described in examples 1-3, with the difference that, on the cover plate, two or three ventrally and/or dorsally and/or laterally offset fixing means are used instead of one centrally mounted fixing means.

Accordingly, the intervertebral disk not only has one recess for accommodating a fixing means, but instead several recesses.

On the bottom plate, for example, two cylindrical pins are mounted in a laterally offset manner. Each pin has a diameter of 4 mm and a height of 4 mm. The intervertebral disk has two round, oval or crescent-shaped recesses which are suitable for accommodating said pins and which are dimensioned in such a way that the intervertebral disk can make translational movements of 1-2 mm in a lateral direction and of 2-6 mm in a ventral-dorsal direction on the base plate.

The two pins limit rotation to about 1.5 degrees.

Also this embodiment enables such motions as those performed in the case of the natural vertebral segment, which can be seen from the course of the IHA or the ICR.

1.-14. (canceled)

15. Intervertebral disk implant, comprising a base plate, a cover plate and an intervertebral disk, wherein the intervertebral disk is seated on the base plate in such a way that translational and rotational movements are possible and said cover plate is seated on the intervertebral disk in such a way that the articulating surface of the intervertebral disk as well as the articulating surface of the cover plate are each located on a respective spherical partial surface with the same radii.

16. Intervertebral disk implant according to claim 15, wherein the intervertebral disk consists of polyethylene or titanium or a titanium alloy.

17. Intervertebral disk implant according to claim 15, wherein the base plate and/or the cover plate can be implanted into the bone or fixed to the bone without cement.

18. Intervertebral disk implant according to claim 16, wherein the base plate and/or the cover plate can be implanted into the bone or fixed to the bone without cement.

19. Intervertebral disk implant according to claim 15, wherein the base plate and/or the cover plate consist of titanium or a titanium alloy.

20. Intervertebral disk implant according to claim 16, wherein the base plate and/or the cover plate consist of titanium or a titanium alloy.

21. Intervertebral disk implant according to claim 17, wherein the base plate and/or the cover plate consist of titanium or a titanium alloy.

22. Intervertebral disk implant according to claim 15, wherein the base plate and/or the cover plate and/or the intervertebral disk of titanium or titanium alloy is coated with a ceramic coating.

23. Intervertebral disk implant according to claim 16, wherein the base plate and/or the cover plate and/or the intervertebral disk of titanium or titanium alloy is coated with a ceramic coating.

24. Intervertebral disk implant according to claim 17, wherein the base plate and/or the cover plate and/or the intervertebral disk of titanium or titanium alloy is coated with a ceramic coating.

25. Intervertebral disk implant according to claim 19, wherein the base plate and/or the cover plate and/or the intervertebral disk of titanium or titanium alloy is coated with a ceramic coating.

26. Intervertebral disk implant according to claim 22, wherein the ceramic coating is titanium-niobium-nitride (Ti—Nb—N).

27. Intervertebral disk implant according to claim 23, wherein the ceramic coating is titanium-niobium-nitride (Ti—Nb—N).

28. Intervertebral disk implant according to claim 24, wherein the ceramic coating is titanium-niobium-nitride (Ti—Nb—N).

29. Use of the intervertebral disk implant according to claim 15 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.

30. Use of the intervertebral disk implant according to claim 16 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.

31. Use of the intervertebral disk implant according to claim 17 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.

32. Use of the intervertebral disk implant according to claim 19 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.

33. Use of the intervertebral disk implant according to claim 22 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.

34. Use of the intervertebral disk implant according to claim 26 for treatment of scoliosis, herniation of intervertebral disk, kyphosis, ruptured disk, black disc, spontaneous deformation, lumbago, deforming spondylosis, age-related hunchback (Witwenbuckel), spondylomyelitis, osteochondrosis, osteofibrosis, spina bifida, lordosis, spondylotosis, clay shoveller's fracture, myelomeningocele, brachialgia, Baastrup's syndrome, vertebral ankylosis, Scheuermann's disease, cervical syndrome, lumbar kyphosis, torticollis, as well as Bechterew's disease.
